(19) United States
(12) Patent Application Publication
(10) Pub. No.: US 2018/0008231 A1
(43) Pub. Date: Jan. 11, 2018

(54) ULTRASOUND PROBE AND THE ULTRASOUND DIAGNOSTIC DEVICE USING SAME

(71) Applicant: Hitachi, Ltd., Tokyo (JP)

(72) Inventors: Tatsuya MIYAKE, Tokyo (JP); Toru WATANABE, Tokyo (JP); Takashi NAITO, Tokyo (JP); Takuya AOYAGI, Tokyo (JP); Taigo ONODERA, Tokyo (JP)

(21) Appl. No.: 15/552,547

(22) PCT Filed: Jan. 27, 2016
(86) PCT No.: PCT/JP2016/052308

§ 371 Date: Aug. 22, 2017

(30) Foreign Application Priority Data
Feb. 27, 2015 (JP) 2015-037602

(51) Int. Cl. A61B 8/14; A61B 8/00; H04R 17/00; H04R 3/00

(52) U.S. Cl. CPC A61B 8/14; H04R 17/00; A61B 8/4444; H04R 3/00

(57) ABSTRACT

An ultrasound probe is provided which maintains sufficient adhesion strength of the layers that configure the ultrasound probe and which matches the acoustic impedance of a piezoelectric element to that of the organism; also provided is an ultrasound diagnostic device provided with said ultrasound probe. This ultrasound probe (100a) is characterized by comprising a backing layer, a piezoelectric element layer (6E), an acoustic matching layer (2A) and an acoustic lens (1), laminated in that order, wherein an adhesion layer (14A) containing vanadium glass is provided between the piezoelectric element layer (6E) and the acoustic matching layer (2A).

### TECHNICAL FIELD

The present invention relates to an ultrasound probe and an ultrasound diagnostic device including the same.

### BACKGROUND ART

In the medical field, ultrasound diagnostic devices are widely used. The ultrasound diagnostic device transmits an ultrasound wave into the organism and receives the ultrasound wave reflected in the organism. Then, based on the received ultrasound wave, image data indicating a tissue in the organism is generated and displayed on a display.

Examples of the image display mode of the ultrasound diagnostic device include a mode for displaying a two-dimensional image (tomographic image), and a mode for displaying a three-dimensional image. The former tomographic image is formed on the basis of frame data (two-dimensional ultrasound data) acquired by one-dimensional scanning of an ultrasound beam, and the latter three-dimensional image is formed based on volume data acquired by two-dimensional scanning of an ultrasound beam.

The ultrasound diagnostic device includes an ultrasound probe which transmits an ultrasound wave according to a given electric signal and outputs an electric signal corresponding to the received ultrasound wave. Examples of the ultrasound probe include an array type ultrasound probe allowing electrical scanning of an ultrasound beam. A plurality of vibration elements are arranged on the array type ultrasound probe. The transmission direction of the ultrasound wave is directed in a specific direction by adjusting the delay times of signals applied to respective vibration elements. By combining signals output from respective vibration elements in accordance with the received ultrasound wave while adjusting the delay times for respective signals, a received signal for the ultrasound wave arriving from the specific direction can be obtained. Accordingly, scanning of the ultrasound beam can be performed by changing the signal delay times for respective vibration elements.

In the case of a 1D array type ultrasound probe performing one-dimensional scanning, an ultrasound beam can be scanned within a scanning plane defined by the direction of vibration elements arranged in a line . Further, in the case of a 2D array type ultrasound probe performing two-dimensional scanning, vibration elements are arranged in a longitudinal direction and a lateral direction, and an ultrasound beam can be scanned in an oblique direction in addition to the longitudinal direction and the lateral direction.

Furthermore, in the case of a 1.5D array type ultrasound probe, vibration elements are arranged in a vertical direction and a horizontal direction as with the 2D array type ultrasound probe. Then, for each set of vibration elements arranged in the vertical direction, predetermined signal delay times are assigned to respective vibration elements arranged in the vertical direction, and an ultrasound beam can be scanned in the scanning plane defined thereby.

FIG. 1A is a perspective view schematically showing an example of the configuration of a conventional ultrasound probe, and FIG. 1B is a cross-sectional view taken along line AB of FIG. 1A. As shown in FIGS. 1A and 1B, an ultrasound probe 100 has a structure in which a piezoelectric element layer 3, an acoustic matching layer 2, and an acoustic lens 1 are laminated in that order on a backing layer 4. In the piezoelectric element layer 3, a plurality of piezoelectric elements (ultrasound oscillators) 6 are two-dimensionally arranged. The piezoelectric element layer 3 is divided into individual piezoelectric elements 6 by separation grooves 7, and the acoustic matching layer 2 is also divided by the separation grooves 7 so as to correspond to the individual piezoelectric elements 6. The piezoelectric element 6 includes a piezoelectric member 9 and electrodes 5 provided on both surfaces of the piezoelectric member 9. A signal line 8 is connected to the lower (backing layer 4 side) electrode 5 through the backing layer 4 made of an insulating member 10, and an ultrasound signal is transmitted and received between the piezoelectric element layer 3 and the backing layer 4. By providing the acoustic lens 1 and the acoustic matching layer 2, an ultrasound wave reflected on a boundary surface between the ultrasound probe and the organism is reduced.

Incidentally, the following PTLs 1 and 2 describe an ultrasound probe in which a plurality of piezoelectric elements are arranged. An acoustic matching layer is laminated on a layer in which the piezoelectric elements are arranged.

### CITATION LIST

### Patent Literature

PTL 1: Japanese Patent Application Laid-Open No. 2014-107853

PTL 2: Japanese Patent Application Laid-Open No. 60-2242

### SUMMARY OF INVENTION

### Technical Problem

FIG. 2A is a cross-sectional view schematically showing an example of the configuration of a conventional ultrasound probe, and FIG. 2B is a graph showing acoustic impedance characteristics of respective layers in FIG. 2A and a matching curve. In FIG. 2A, only one piezoelectric element and an acoustic matching layer provided thereon are shown for easy viewing. Further, in the drawings, a piezoelectric member and an electrode are not distinguished, and these are combined to form a piezoelectric element. Hereinafter, the same applies to FIGS. 3A to 7A.

An acoustic matching layer 2 usually includes two layers or three or more layers. FIGS. 2A and 2B show an example in which the acoustic matching layer 2 includes three layers (2A, 2B, 2C). As shown in FIG. 2B, in general, the acoustic impedances of respective layers configuring the acoustic matching layer 2 are adjusted so as to follow a matching curve 13 decreasing exponentially from the organism 12 toward a piezoelectric element 6E in order to reduce the reflection of an ultrasound wave. However, the adjacent two layers of the acoustic matching layers 2A to 2C, the acoustic matching layer 2A and the piezoelectric element 6E, and the acoustic lens 1 and the acoustic matching layer 2C are bonded by adhesion layers (11A, 11B, 11C, 11D). Since an adhesive such as epoxy based adhesive is used for the adhesion layer, the acoustic impedances of respective adhesion layers depart from the matching curve 13 as shown in FIG. 2B, and the reflection of an ultrasound signal therein increases, which may cause signal attenuation. In the future, in order to improve diagnostic performance (resolution performance, depiction performance of a deep part) by an ultrasound probe, it is necessary to reduce the signal attenuation in the adhesion layer.

The bonding of respective layers requires strength for withstanding impact during separation processing. Weak bonding strength causes a decrease in the manufacturing yield of the ultrasound probe.

In the above-mentioned PTLs 1 and 2, sufficient consideration has not been made in order to achieve both acoustic impedance matching between the organism and the piezoelectric element layer and bonding strength between the layers configuring the ultrasound probe.

In light of the above-mentioned circumstances, an object of the present invention is to provide an ultrasound probe which maintains sufficient adhesion strength of layers configuring the ultrasound probe and matches the acoustic impedance of the organism to that of a piezoelectric element, and an ultrasound diagnostic device including the same.

### Solution to Problem

In order to achieve the above object, the present invention provides an ultrasound probe including: a backing layer; a piezoelectric element layer; an acoustic matching layer; and an acoustic lens, the backing layer, the piezoelectric element layer, the acoustic matching layer, and the acoustic lens laminated in that order, wherein an adhesion layer containing vanadium glass is provided between the piezoelectric element layer and the acoustic matching layer.

In order to achieve the above object, the present invention provides an ultrasound diagnostic device including: a transmission beamformer for causing an ultrasound probe to generate a transmission signal at a timing required for forming a focus; a receiving beamformer for converting an ultrasound wave received by the ultrasound probe into an electric signal and subjecting the electric signal to temporal delay to obtain an ultrasound beam signal; a signal processing circuit for extracting a frequency component required for imaging from the ultrasound beam signal and subjecting the frequency component to detection-logarithmic compression in order to convert the frequency component into image luminance information, thereby obtaining an image signal on a scan line; a scan converter for converting the obtained image signal into a digital signal and subjecting all scan lines to work for storing the digital signal at a place corresponding to a position of a scan line in a frame memory to configure an image; and a monitor for displaying the image, wherein the ultrasound probe is the above-mentioned ultrasound probe according to the present invention.

### Advantageous Effects of Invention

The present invention can provide an ultrasound probe which maintains sufficient adhesion strength of layers configuring the ultrasound probe and matches the acoustic impedance of the organism to that of a piezoelectric element, and an ultrasound diagnostic device including the same. By matching the acoustic impedance of the organism to that of the piezoelectric element, it is possible to improve diagnostic performance (resolution performance, observation performance of a deep part) and shorten a diagnosis time. Further, by maintaining the sufficient adhesion strength of the layers, the manufacturing yield of the ultrasound probe can be improved.

### BRIEF DESCRIPTION OF DRAWINGS

FIG. 1A is a perspective view schematically showing an example of the configuration of a conventional ultrasound probe.

FIG. 1B is a cross-sectional view taken along line AB of FIG. 1A.

FIG. 2A is a cross-sectional view schematically showing an example of the configuration of a conventional ultrasound probe.

FIG. 2B is a graph showing acoustic impedance characteristics of respective layers in FIG. 2A and a matching curve.

FIG. 3A is a cross-sectional view schematically showing a part of the configuration of an ultrasound probe in first example of the present invention.

FIG. 3B is a graph showing acoustic impedance characteristics of respective layers in FIG. 3A and a matching curve.

FIG. 4A is a cross-sectional view schematically showing a part of the configuration of an ultrasound probe in second example of the present invention.

FIG. 4B is a graph showing acoustic impedance characteristics of respective layers in FIG. 4A and a matching curve.

FIG. 5A is a cross-sectional view schematically showing a part of the configuration of an ultrasound probe in third example of the present invention.

FIG. 5B is a graph showing acoustic impedance characteristics of respective layers in FIG. 5A and a matching curve.

FIG. 6A is a cross-sectional view schematically showing a part of the configuration of an ultrasound probe in fourth example of the present invention.

FIG. 6B is a graph showing acoustic impedance characteristics of respective layers in FIG. 6A and a matching curve.

FIG. 7A is a cross-sectional view schematically showing a part of the configuration of an ultrasound probe in fifth example of the present invention.

FIG. 7B is a graph showing acoustic impedance characteristics of respective layers in FIG. 7A and a matching curve.

FIG. 8 is a block diagram showing an example of the configuration of an ultrasound diagnostic device including an ultrasound probe according to the present invention.

FIG. 9 is a graph showing the relationship between the viscosity of glass and temperature.

FIG. 10 is a differential thermal analysis graph of glass.

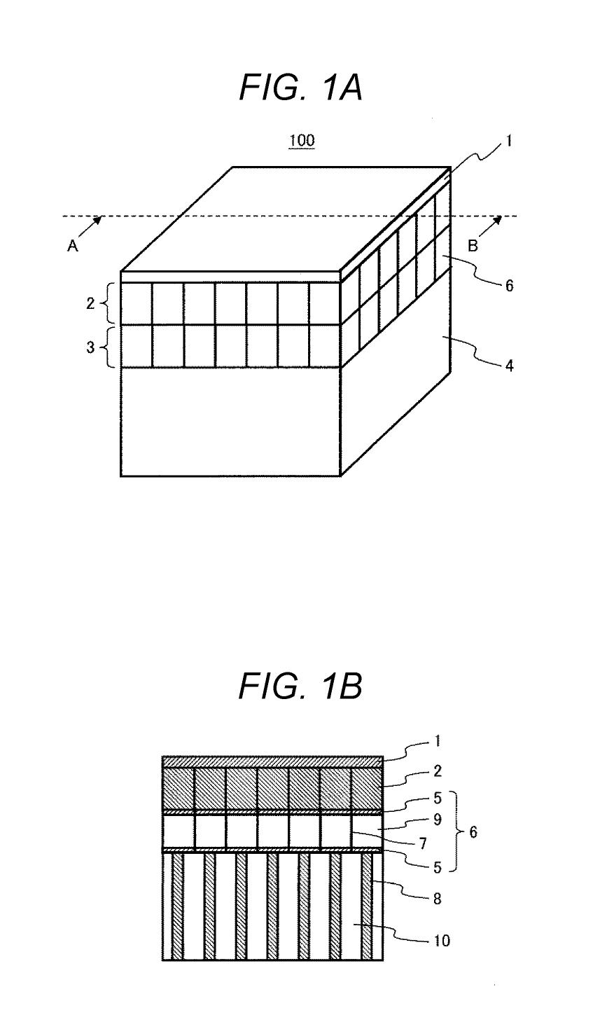

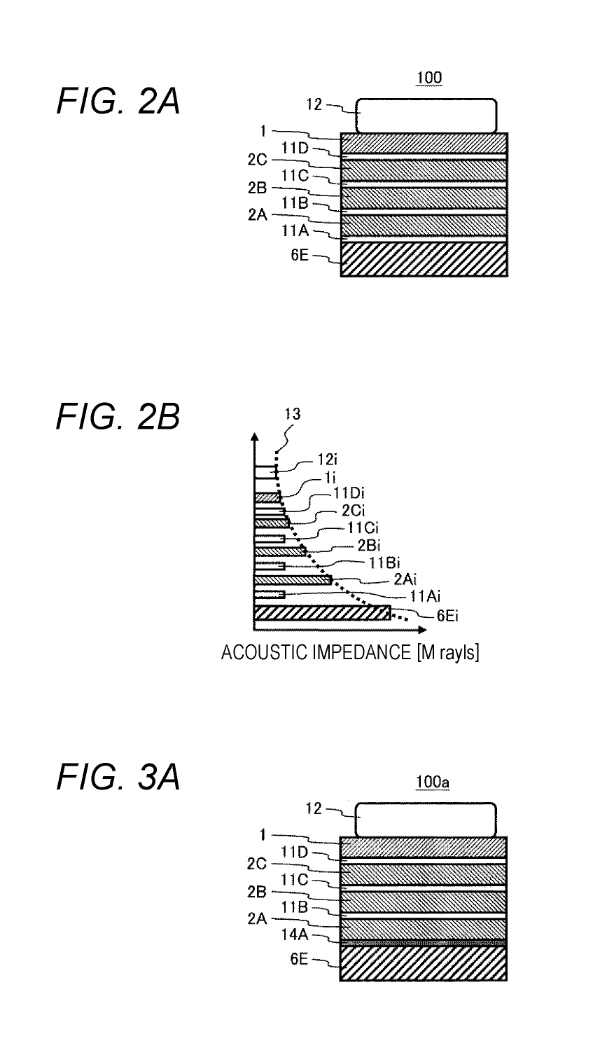

### DESCRIPTION OF EMBODIMENTS

Hereinafter, embodiments of the present invention will be described in detail with reference to the drawings. However, the scope of the present invention is not limited to the following examples. In the following description, components having the same functions and configurations are given the same reference numerals, and the description once described will not be repeated after the second time.

### EXAMPLE 1

FIG. 3A is a cross-sectional view schematically showing a part of the configuration of an ultrasound probe according to first example of the present invention, and FIG. 3B is a graph showing acoustic impedance characteristics of respective layers in FIG. 3A and a matching curve. For convenience of description, an organism 12 is also illustrated together with the configuration of the ultrasound probe besides the structure of the ultrasound probe in FIG. 3A, and the same applies to FIGS. 3A to 7A to be described later. In addition, in FIG. 3B, “6Ei” shows the acoustic impedance of a piezoelectric element layer 6E, and the same applies to the other layers and FIGS. 3A to 7A to be described later.

In an ultrasound probe 100a according to the present example, lead zirconate titanate (hereinafter referred to as PZT) as a piezoelectric ceramic was used as a piezoelectric member configuring a piezoelectric element 6E. Vanadium glass was applied as an adhesive 14A in order that the acoustic impedance of an adhesion layer 14A adhering the piezoelectric element 6E to a first acoustic matching layer 2A which was an acoustic matching layer closest to the piezoelectric element 6E followed s a matching curve 13. The acoustic impedance of PZT is about 35 Mrayls, and the acoustic impedance of the vanadium glass is about 15 Mrayls. By using the above materials as the piezoelectric element 6E and the adhesion layer 14A, the acoustic impedances of the piezoelectric element 6E and the adhesion layer 14A can be made to follow the matching curve 13, which can reduce the attenuation of an ultrasound signal.

The difference in thermal expansion coefficient between the piezoelectric element 6E and the adhesion layer 14A is preferably as small as possible from the viewpoint of adhesion strength. In this respect, since the thermal expansion coefficient of PZT is 5 to 10 ppm/K and the thermal expansion coefficient of the vanadium glass is 7 to 9 ppm/K, the matching of the thermal expansion coefficients of both the materials is good and sufficient adhesion strength is obtained. The thermal expansion coefficient of the vanadium glass can be adjusted by the type and concentration of an additive component (various oxides or filler materials to be described later) to be added to the vanadium glass.

Considering the heat resistant temperature (unpolarizing temperature) of PZT, the softening point of the vanadium glass to be applied to the adhesion layer 14A is preferably 450° C. or less. The softening point of the vanadium glass can be adjusted by an additive (for example, P₂O₅). In the present example, low melting point glass (containing barium, phosphorus, and antimony as an additive element) having a softening point of 445° C. was used.

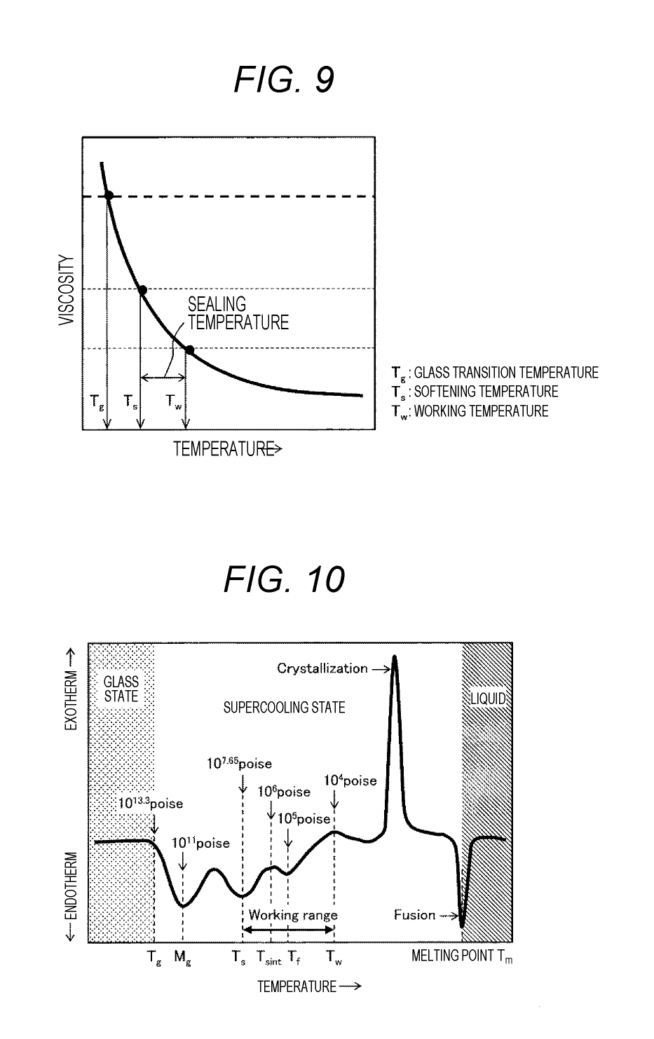

Here, the definition of the softening point of the present invention will be described below. FIG. 9 is a graph showing the relationship between the viscosity of glass and temperature, and FIG. 10 is a differential thermal analysis (DTA) graph of glass. DTA measurement was carried out using α-alumina as a reference sample at a rate of temperature rise of 5° C./min in the atmosphere. The masses of the reference sample and measurement sample were 650 mg.

As shown in FIG. 9, as the temperature of glass increases, the viscosity thereof decreases. Further, in the present invention, as shown in FIG. 10, the starting temperature of a first endothermic peak (temperature at which glass changes to supercooled liquid) is defined as a glass transition point T_{g} (corresponding to viscosity=10^{13.3} poise) ; the peak temperature of the first endothermic peak (temperature at which the expansion of glass stops) is defined as a yield point M_{g} (corresponding to viscosity=10¹¹ poise); the peak temperature of a second endothermic peak (temperature at which glass begins to soften) is defined as a softening point Tₛ (viscosity=10^{7.65} poise); a temperature at which glass becomes a sintered body is defined as a sintering point Tₛᵢₙₜ (corresponding to viscosity =10⁶ poise); a temperature at which glass melts is defined as a flow point T_{f} (corresponding to viscosity=10⁵ poise); and a temperature suitable for forming glass (temperature at which viscosity is 10⁴ dPas) is defined as a working point T_{w}. Each temperature shall be a temperature determined by a tangent method. The softening point Tₛ described herein is based on the above definition.

The transition point T_{g} and the softening point Tₛ are values such as 373° C. and 445° C., and vanadium glass heated at a temperature in a range of the softening point to a working point can act as an adhesive.

The vanadium glass can be prepared by adding phosphorus (P) or the like as a vitrifying component to vanadium pentoxide (V₂O₅) to obtain a mixture and melting the mixture. The addition amount of V₂O₅ is preferably 20 to 70 vol %, and more preferably 40 to 60 vol %. The addition amount of V₂O₅ of less than 20 vol % provides an insufficient effect of the vanadium glass (matching of acoustic impedance and thermal expansion coefficient with those of the piezoelectric element 6E). The addition amount of V₂O₅ exceeding 70 vol % excessively increases the acoustic impedance. The acoustic impedance deviates from the matching curve 13. The addition amount of V₂O₅ exceeding 70 vol % causes voids of air generated in a material, which attenuates an acoustic signal itself, to decrease the resolution of the ultrasound probe.

The vanadium glass contains the above vanadium glass as a main component. The vanadium glass may contain various elements as additives if necessary. For example, the vanadium glass may contain phosphorus (P) which is a vitrifying component, antimony (Sb), barium (Ba), or iron (Fe) which is a water resistance improving component, manganese (Mn), tellurium (Te), sodium (Na), potassium (K), zinc (Zn), or tungsten (W) which is a glass stabilizing component, or the like.

The above elements can be added to the vanadium glass in forms of phosphorus pentoxide (P₂O₅), antimony trioxide (Sb₂O₃), barium oxide (BaO), iron (III) oxide (Fe₂O₃), manganese (II) oxide (MnO), manganese dioxide (MnO₂) tellurium dioxide (TeO₂), sodium oxide (Na₂O), potassium oxide (K₂O), ZnO (zinc oxide) and tungsten oxide (WO₃) or the like.

In order to apply the vanadium glass on the piezoelectric element 6E, the vanadium glass is made paste. There is no particular limitation on a method of preparing the paste. For example, the paste can be prepared by mixing ethyl cellulose and diethylene glycol monobutyl ether acetate with the vanadium glass in a kneader, followed by performing a vacuum defoaming treatment.

The piezoelectric element 6E and the acoustic matching layer 2A can be bonded to each other by applying the paste on the piezoelectric element 6E, placing the acoustic matching layer 2A on the piezoelectric element 6E to form a laminate, and heating the laminate at a temperature of 450 to 500° C. for 15 minutes.

In the present example, after the piezoelectric member 6E and the first acoustic matching layer 2A are bonded with the adhesion layer 14A, a backing layer (not shown) is bonded to a lower portion of the piezoelectric element 6E, and the subsequent second acoustic matching layer 2B was bonded to an upper portion of the acoustic matching layer 2A to manufacture the ultrasound probe. For adhesion layers 11B to 11D, a conventional epoxy resin adhesive was used.

In the present example, the materials of respective acoustic matching layers were selected so that the acoustic impedance characteristics of respective layers were the acoustic impedance characteristics shown in FIG. 3B. A material having a thermal expansion coefficient of 9.3 ppm/K was used as the first acoustic matching layer 2A. The thermal expansion coefficient a of the vanadium glass paste is 7.8 ppm/K, which is about the same as the thermal expansion coefficient of PZT (a: 5 to 10 ppm/K) and the thermal expansion coefficient of the first acoustic matching layer 2A. Therefore, for bonding between the piezoelectric element 6E and the first acoustic matching layer 2A, bonding strength with a shear stress of 10 kgf/mm² or more was obtained, and a processing yield during element cutting was also good.

Examples of glasses having an acoustic impedance of about 15 Mrayls include Pb (lead)-based glass and Bi (bismuth)-based glass in addition to vanadium glass, but the use of the Pb-based glass is inappropriate as it is environmentally harmful. Since the Bi (bismuth)-based glass has a softening point of higher than 600° C. and a thermal expansion coefficient of 10 to 12 ppm, and the difference between the Bi (bismuth)-based glass and PZT is larger than the difference between the vanadium glass and PZT, the Bi (bismuth)-based glass is not preferable considering the heat resistance temperature of PZT and the bonding strength of the ultrasound probe.

The piezoelectric member 9 configuring the piezoelectric element 6E is not limited to the above-described PZT, and various piezoelectric materials can be used. For example, as an inorganic piezoelectric material, thin films made of quartz, piezoelectric ceramics such as PZT, (Pb, La) (Zr, Ti)Oₓ perovskite compound (PZLT), and piezoelectric single crystals such as lead niobate zirconate-lead titanate solid solution (PZN-PT), lead magnesium niobate-lead titanate solid solution (PMN-PT), lithium niobate (LiNbO₃), lithium tantalate (LiTaO₃), potassium niobate (KNbO₃), zinc oxide (ZnO) and aluminum nitride (AlN) can be used. Examples of an organic piezoelectric material include polyvinylidene fluoride, polyvinylidene fluoride copolymers, polyvinylidene polyanide, vinylidene cyanide copolymers, odd nylons such as nylon 9 and nylon 11, aromatic nylons, alicyclic nylons, polylactic acid, polyhydroxycarboxylic acid such as polyhydroxybutyrate, cellulose derivatives, and polyurea. Further, a composite material including the inorganic piezoelectric material and the organic piezoelectric material in combination, or including the inorganic piezoelectric material and an organic polymer material in combination can also be used. The acoustic impedance of the piezoelectric material is about 20 to 40 Mrayls, and the thermal expansion coefficient is about 5 to 10 ppm/K which is the same as that of PZT. With respect to the heat resistance of the piezoelectric body, the adhesion treatment temperature (450 to 500° C.) of vanadium glass having a softening point of 450° C. or lower has no problem.

As the constituent materials of the acoustic matching layers 2A to 2C, aluminum (Al), aluminum alloys such as aluminum-magnesium (Al—Mg) alloys, magnesium alloys, glass, fused quartz, polyethylene (PE), polypropylene (PP), polycarbonate (PC), acrylonitrile-butadiene-styrene resins (ABC resins), acrylonitrile-butadiene-styrene copolymerization synthetic resins (ABS resins), acrylonitrile-acrylic ester-styrene copolymerization synthetic resins (AAS resins), acrylonitrile-ethylene-propylene-diene-styrene copolymerization synthetic resins (AES resins), nylon (PA6, PA 6-6), polyphenylene oxide (PPO), polyphenylene sulfide (PPS, may contain a glass fiber), polyphenylene ether (PPE), polyetheretherketone (PEEK), polyamide imide (PAI), polyethylene terephthalate (PETP), epoxy resins, and urethane resins or the like can be used. Preferably, a molded product obtained by adding zinc oxide (ZnO), titanium oxide (TiO₂). silica (SiO₂), alumina (Al₂O₃), red iron oxide, ferrite, tungsten oxide (WO₂), yttrium oxide (Y₂O₃), barium sulfate (BaSO₄), tungsten (W), and molybdenum (Mo) or the like as a filler to a thermosetting resin such as an epoxy resin, and followed by molding can be applied.

An acoustic lens 1, a backing layer 4 and an electrode 5 are not particularly limited, and conventional materials can be used therefor. For the acoustic lens 1, silicone rubber or the like is mainly used. As the backing layer 4, an epoxy resin filled with metal powder, and rubber filled with filament powder, or the like are used. As the electrode 5, a gold electrode or the like is mainly used.

### EXAMPLE 2

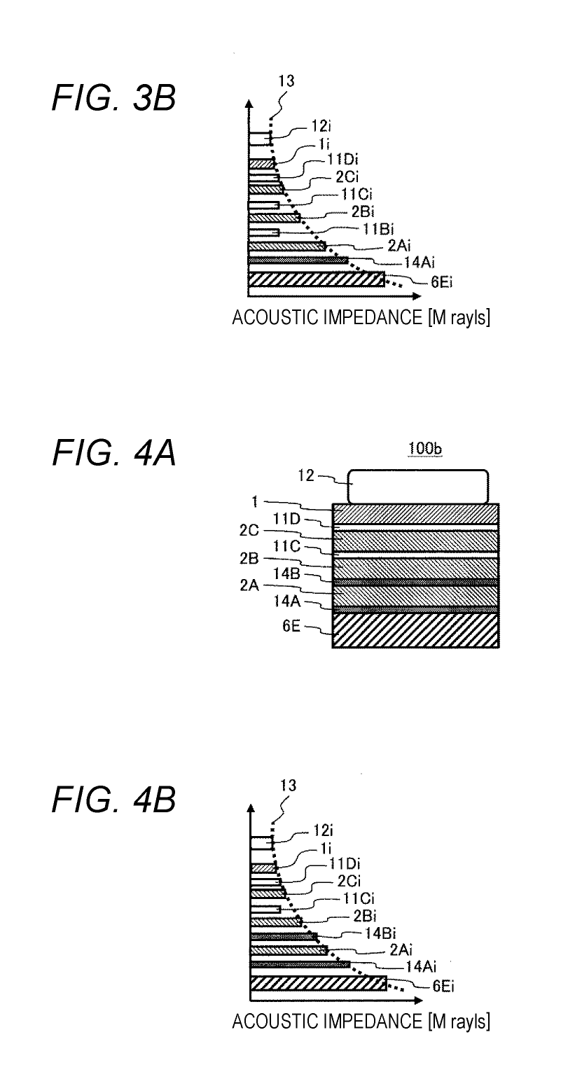

In example 1, the vanadium glass was applied only to the adhesion layer 14A between the piezoelectric member 6E and the first acoustic matching layer 2A. However, in the present example, an example in which vanadium glass is applied also to an adhesion layer 14B between a first acoustic matching layer 2A and a second acoustic matching layer 2B will be described with reference to FIGS. 4A and 4B.

FIG. 4A is a cross-sectional view schematically showing a part of the configuration of an ultrasound probe according to second example of the present invention, and FIG. 4B is a graph showing acoustic impedance characteristics of respective layers in FIG. 4A and a matching curve. Since the acoustic impedance of the first acoustic matching layer 2A used in the present example is about 15 Mrayls, from the viewpoint of acoustic impedance matching, it is appropriate that the adhesion layer 14B has an acoustic impedance of about 12 to 13 Mrayls. Therefore, the acoustic impedance was lowered from about 15 Mrayls to about 12 Mrayls by adding 20 vol % of silica (SiO₂) powder (average particle diameter: 10 μm) as a filler material to the vanadium glass used for the adhesion layer 14A in Example 1, to allow acoustic impedance characteristics following the matching curve to be obtained, as shown in FIG. 4B.

As described above, the acoustic impedance of the adhesion layer 14B can be adjusted not only by adjusting the additive of the vanadium glass but also by adding the filler material to the vanadium glass. The acoustic impedance can be adjusted by adjusting the addition amount of the filler material. As the filler material, alumina (Al₂O₃) and silica (SiO₂) can be preferably used. Since alumina is heavier (has a larger mass number) than vanadium glass, the alumina is preferably added when the acoustic impedance is set to be larger than that of the vanadium glass . Since silica is lighter (has a smaller mass number) than the vanadium glass, the silica is preferably added when the acoustic impedance is set to be larger than that of the vanadium glass . Material cost can be reduced by adding the relatively inexpensive filler material in place of the vanadium glass.

A method of preparing the adhesion layer 14B to which the filler material is added is not particularly limited, but the adhesion layer 14B can be produced by, for example, adding a finely powdered filler material to finely powdered vanadium glass, followed by powder compacting.

### EXAMPLE 3

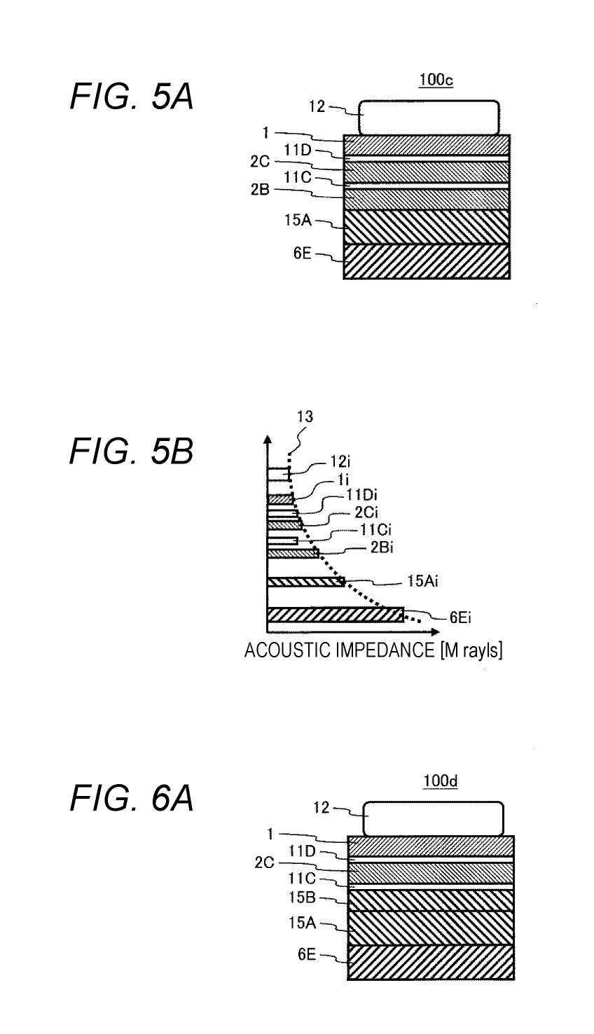

FIG. 5A is a cross-sectional view schematically showing a part of the configuration of an ultrasound probe in third example of the present invention, and FIG. 5B is a graph showing acoustic impedance characteristics of respective layers in FIG. 5A and a matching curve. In the present example, an example in which vanadium glass is applied to three layers (the first acoustic matching layer 2A, the adhesion layer 14A and the adhesion layer 11B) in example 1 will be described with reference to FIGS. 5A and 5B.

A glass sheet (plate thickness: 100 μm) made of vanadium glass as a first acoustic matching layer 15A is inserted between a piezoelectric member 6E (PZT) and a second acoustic matching layer 2B, followed by bonding. The bonding was carried out by thinly applying a vanadium glass paste having the same composition as that of the glass sheet on the upper and lower surfaces of the acoustic matching layer 15A, and laminating the piezoelectric element 6E and the acoustic matching layer 2B, followed by firing.

Since the three layers can realized with one material (vanadium glass) in the present example, process cost can be reduced. In terms of the characteristics of the acoustic matching layer, the attenuation of the ultrasound signal in these layers was small, and the bonding strength could also be increased.

### EXAMPLE 4

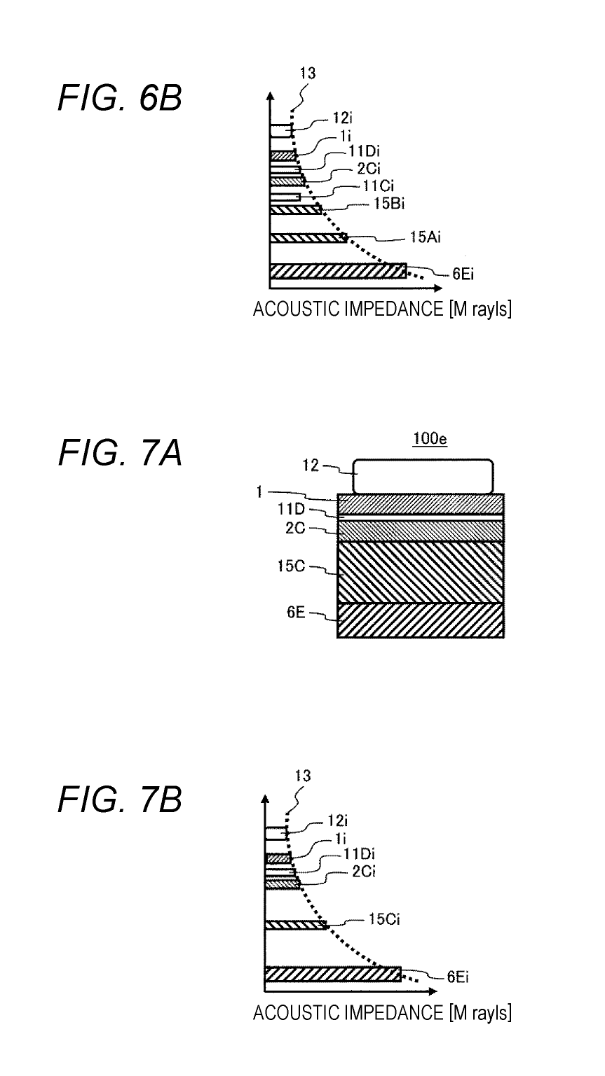

FIG. 6A is a cross-sectional view schematically showing a part of the configuration of an ultrasound probe in fourth example of the present invention, and FIG. 6B is a graph showing acoustic impedance characteristics of respective layers in FIG. 6A and a matching curve. In the present, an example in which vanadium glass is applied to the acoustic matching layer 2B in example 3 will be described with reference to FIGS. 6A and 6B.

In order to apply vanadium glass to the acoustic matching layer 2B, it is necessary to lower the acoustic impedance to about 10 Mrayls. Therefore, by adding 40 vol % silica (SiO₂) powder (average particle diameter: 10 μm) as a filler material to the vanadium glass, the acoustic impedance was reduced to about 10 Mrayls, to form an acoustic matching layer 15B. Since an acoustic matching layer 15A and the acoustic matching layer 15B which included the vanadium glass were bonded to each other, the acoustic matching layer 15A and the acoustic matching layer 15B could be bonded by firing at 450° C. or more in a state where flat surfaces of both the layers were exposed.

Since the four layers can be realized with the vanadium glass according to the present example, process cost can be reduced. In terms of the characteristics of the acoustic matching layer, the attenuation of the ultrasound signal in these layers was small, and the bonding strength could also be improved.

### EXAMPLE 5

FIG. 7A is a cross-sectional view schematically showing a part of the configuration of an ultrasound probe according to fifth example of the present invention, and FIG. 7B is a graph showing acoustic impedance characteristics of respective layers in FIG. 7A and a matching curve. In Examples 1 to 4, the acoustic matching layers 2 including three layers were used (three-layer model). In the present example, an aspect in which vanadium glass is applied to a two-layer model will be described with reference to FIGS. 7A and 7B.

In the present example, the acoustic impedance was reduced to about 10 Mrayls by the addition of a filler material in the same manner as the method of the acoustic matching layer 15B of example 4, and a first acoustic matching layer 15C shown in FIG. 7 was applied. A second acoustic matching layer 2C was bonded to the upper part of the acoustic matching layer 15C, thereby manufacturing an ultrasound probe. The number of components of the present example was less than that of the three-layer model, which could achieve a reduction in cost and an improvement in bonding strength.

### EXAMPLE 6

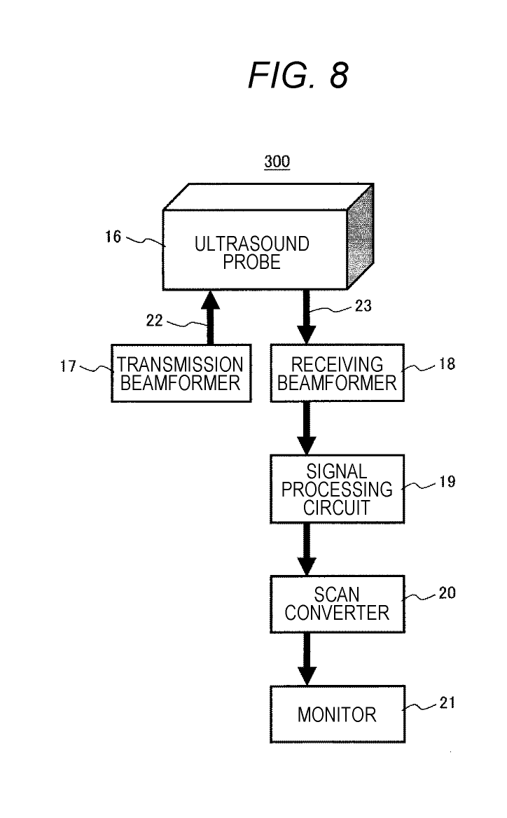

FIG. 8 is a block diagram showing an example of the configuration of an ultrasound diagnostic device including an ultrasound probe according to the present invention. In the present, examples in which ultrasound diagnostic devices (an ultrasound pulse reflection method is applied) are configured using the respective ultrasound probes of examples 1 to 5 will be described with reference to FIG. 8.

As shown in FIG. 8, an ultrasound diagnostic device 300 includes: an ultrasound probe 16 for generating and detecting an ultrasound wave; a transmission beamformer 17 for causing the ultrasound probe 16 to generate a transmission signal 22 at a timing required for forming a focus; a receiving beamformer 18 for converting an ultrasound wave received by the ultrasound probe 16 into an electric signal 23 and subjecting the electric signal to temporal delay to obtain an ultrasound beam signal; a signal processing circuit 19 for extracting a frequency component required for imaging from the obtained beam signal and subjecting the frequency component to detection-logarithmic compression in order to convert the frequency component into image luminance information, thereby obtaining an image signal on a scan line; a scan converter 20 for converting the obtained image signal into a digital signal and subjecting all scan lines to work for storing the digital signal at a place corresponding to a position of a scan line in a frame memory to configure an image; and a monitor 21 for displaying the image.

In the present example, by using the ultrasound probes of examples 1 to 5 as the ultrasound probe 16, the acoustic impedance matching properties of respective layers configuring the ultrasound probe are improved, which can provide an ultrasound diagnostic device making it possible to improve diagnostic performance (resolution performance, deep part) and shorten a diagnosis time.

As described above, it was proved that the present invention can provide an ultrasound probe which maintains sufficient adhesion strength of respective layers configuring an ultrasound probe and matches the acoustic impedance of the organism to that of a piezoelectric element, and an ultrasound diagnostic device including the same.

The present invention is not limited to the above-described examples, and includes various modifications. For example, the above-described examples are described in detail for convenience of description and good understanding of the present invention, and thus the present invention is not limited to one including all the described configurations. In the present invention, it is possible to delete some of the configurations of embodiments and examples in the present specification, replace some of the configurations by the other configurations, and add the other configurations to some of the configurations.

### REFERENCE SIGNS LIST

1 acoustic lens
2 acoustic matching layer
2A first acoustic matching layer
2B second acoustic matching layer
2C third acoustic matching layer
3 piezoelectric element layer
4 backing layer
5 electrode
6, 6E piezoelectric element
9 piezoelectric member
7 separation groove
8 signal line
10 insulating material
11A, 11B, 11C, 11D adhesion layer
12 organism
13 matching curve
14A, 14B vanadium glass adhesion layer
15A, 15B, 15C vanadium glass acoustic matching layer
17 transmission beamformer
18 receiving beamformer
19 signal processing circuit
20 scan converter
21 monitor
22 transmission signal
23 ultrasound signal
16, 100, 100a, 100b, 100c, 100d, 100e ultrasound probe
300 ultrasound diagnostic device

1. An ultrasound probe comprising:
a backing layer;
a piezoelectric element layer;
an acoustic matching layer; and
an acoustic lens,
the backing layer, the piezoelectric element layer, the acoustic matching layer, and the acoustic lens laminated in that order,
wherein an adhesion layer containing vanadium glass is provided between the piezoelectric element layer and the acoustic matching layer.

2. The ultrasound probe according to claim 1, wherein
the ultrasound probe has a structure in which the plurality of acoustic matching layers are laminated, and
an adhesion layer containing vanadium glass is provided between at least any two of the adjacent acoustic matching layers.

3. The ultrasound probe according to claim 2, wherein
the acoustic matching layer has a structure in which a first acoustic matching layer, an adhesion layer, and a second acoustic matching layer are laminated in that order on the piezoelectric element layer, and
the adhesion layer provided between the first acoustic matching layer and the second acoustic matching layer contains vanadium glass.

4. The ultrasound probe according to claim 3, wherein the first acoustic matching layer contains vanadium glass.

5. The ultrasound probe according to claim 3, wherein the first acoustic matching layer and the second acoustic matching layer contain vanadium glass.

6. The ultrasound probe according to claim 2, wherein the acoustic matching layer comprises two layers.

7. The ultrasound probe according to claim 1, wherein the vanadium glass has a softening point of 450° C. or less, a thermal expansion coefficient of 7 to 9 ppm/K, and an acoustic impedance of 15 Mrayls.

8. The ultrasound probe according to claim 1, wherein the vanadium glass contains alumina or silica as a filler material.

9. The ultrasound probe according to claim 1, wherein the vanadium glass contains at least one of phosphorus, antimony, barium, iron, manganese, tellurium, sodium, potassium, zinc, and tungsten.

10.The ultrasound probe according to claim 1, wherein
the piezoelectric element layer is formed by two-dimensionally arranging a plurality of piezoelectric elements, and
the piezoelectric elements contain lead zirconate titanate.

11. An ultrasound diagnostic device comprising:
a transmission beamformer for causing an ultrasound probe to generate a transmission signal at a timing required for forming a focus;
a receiving beamformer for converting an ultrasound wave received by the ultrasound probe into an electric signal and subjecting the electric signal to temporal delay to obtain an ultrasound beam signal;
a signal processing circuit for extracting a frequency component required for imaging from the ultrasound beam signal and subjecting the frequency component to detection-logarithmic compression in order to convert the frequency component into image luminance information, thereby obtaining an image signal on a scan line;
a scan converter for converting the obtained image signal into a digital signal and subjecting all scan lines to work for storing the digital signal at a place corresponding to a position of a scan line in a frame memory to configure an image; and
a monitor for displaying the image,
wherein the ultrasound probe is the ultrasound probe according to claim 1.
